(19) United States
(12) Patent Application Publication
(10) Pub. No.: US 2007/0244366 A1
(43) Pub. Date: Oct. 18, 2007

(54) Electronic endoscope

(75) Inventor: Masanao Murata, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/585,541

(22) Filed: Oct. 24, 2006

(30) Foreign Application Priority Data
Nov. 8, 2005 (JP) 2005-324009

(51) Int. Cl. A61B 1/005

(52) U.S. Cl. 600/175

(57) ABSTRACT

A white LED portion of an optical adapter is configured by a plurality of LED elements in an n-serial and m-parallel matrix configuration, and an identification resistance for identifying the type of the white LED portion. In an operation portion of an endoscope portion, an LED drive portion for driving the white LED portion is provided, and the LED drive portion is configured by an LED drive circuit and a VR unit. An electronic endoscope of the invention can be detachably attached with different optical adapters each having an LED illumination portion, and drives the LED illumination portion under the optimum driving condition.

### CROSS REFERENCE TO RELATED APPLICATIONS

This application claims benefit of Japanese Application No. 2005-324009 filed in Japan on Nov. 8, 2005, the contents of which are incorporated by this reference.

### BACKGROUND OF THE INVENTION

1. Field of the Invention

The present invention relates to an electronic endoscope in which an adapter is equipped with at least an illumination power source unit, and the adapter is equipped to the distal end of an insertion portion.

2. Description of the Related Art

In recent years, such endoscopes have been widely used that inserting an elongate insertion portion into a body cavity allows observing intracavital organs and the like, and using a therapeutic device introduced into a therapeutic device channel as needed permits performing various medical treatments. Also in the industrial field, industrial endoscopes are widely used for observing and inspecting internal abrasions, corrosions, and so forth, of a boiler, turbine, engine, chemical plant, and the like.

Endoscopes used as mentioned above include an electronic endoscope (hereinafter abbreviated as “endoscope”) having an insertion portion provided at the distal end thereof with an image sensor such as a CCD for photoelectrically converting an optical image into an image signal. This endoscope is configured so that an image signal of an observation image formed on the image sensor is transmitted to a signal processing unit of a camera control unit (hereinafter abbreviated as “CCU”) which is an external device for generating a video signal, and then the endoscope image is displayed on a monitor screen to perform an observation.

In recent years, to address various inspections, for the industrial endoscopes are available various optical adapters that are exchangeably attached to the distal end of the endoscope, so as to cope with diameter dimensional differences and inspection purposes, the dimensional difference being due to various small to large diameters of the observation targets such as pipes.

For example, Japanese unexamined patent publication No. 2001-61777 proposes an electronic endoscope having an operation portion including: a video output terminal portion for outputting to a display unit a video signal outputted from the image sensor; and a current control circuit for controlling a current value to be supplied to the illumination LED, the operation portion being equipped to the proximal end of an insertion portion which has at its distal end a detachable image-capturing adapter equipped with an image sensor and an illumination LED.

Here, an example of an endoscope device having a conventional industrial endoscope will be described using FIG. 12.

As shown in FIG. 12, a conventional industrial endoscope device 100 has an elongate and flexible insertion portion, and is configured by: an endoscope 101 having in the distal end a CCD 106 serving as an image sensor; an optical adapter 102 with an in-built white LED 104, serving as an illumination portion detachably provided to the distal end of the endoscope 101; and a main body 103 for driving the white LED 104 and signal processing an output signal from the CCD 106 to generate a video signal so as to display an endoscope image on a monitor 110.

The optical adapter 102 includes, in addition to the white LED 104, an object optical system 105 for forming on the image-capturing surface of the CCD 106 an image of a subject to which illumination light from the white LED 104 is irradiated, and an identification resistance R1 for identifying the type of the optical adapter 102.

The main body 103 includes a CCD driver 109 for driving the CCD 106, a video signal processing circuit 111 for signal processing an output signal from the CCD 106 and outputting a video signal to the monitor 110, an LED drive circuit 115 for driving the white LED 104, and so forth.

In the main body 103, the identification resistance R1 is detected by an ID detection unit 112, and by means of the detection signal, a CPU 113 identifies the type of the optical adapter 102, i.e., the type of the white LED 104. Note that the white LED 104 can have different configurations depending on the optical adapter 102, as will be described later.

The CPU 113 controls circuits in the endoscope device, such as the CCD driver 109, the video signal processing circuit 111, and the LED drive circuit 115, according to a program pre-stored in a ROM 114.

The main body 103 is configured as a carriable device, in that the main body 103 is supplied with an electric power by a battery 107, and a power supply unit 108 generates the electric power of a circuit voltage Vdd in the device.

Note that in the main body 103, the CPU 113 can transmit data such as an endoscope image to a memory card 113 and a personal computer (PC) 117, thereby saving these data thereinto.

To refer to the configuration of FIG. 12, the endoscope of the above-mentioned Japanese unexamined patent publication No. 2001-61777 is configured so that the white LED 104 and the CCD 106 are provided in the optical adapter 102, and the CCD driver 109 and the LED drive circuit 115 are provided in the operation portion provided at the proximal end of the insertion portion of the endoscope.

The white LED 104 provided to the optical adapter 102 is not limited to the configuration of FIG. 12, but may be configured as in FIGS. 13 to 16. Then, by the identification resistance having different resistance values depending on the configurations, the CPU 113 identifies the type of the optical adapter 102, i.e.; the type of the white LED 104.

Here, the types of the white LED 104 are described. The white LED 104 provided to the optical adapter 102 has a matrix configuration with white LED elements in an n-serial and m-parallel arrangement to obtain a light amount needed for illumination, as shown in FIG. 17. If this matrix configuration is expressed as, e.g., D (nS, mP), then in the configurations of FIGS. 12 to 15, the white LED 104 has matrix configurations of D (1S, 1P), D (1S, 2P), D (2S, 2P), D (2S, 3P), and D (3S, 2P), providing one, two, four, six, and six LED illuminations, respectively.

Note that, as shown in FIG. 18, developments in semiconductor technology in recent years have improved the efficiency of LED light emission amount (light beam) with respect to a drive current (NewLED vs. OldLED in FIG. 18), thus requiring a drive current adjustment.

### SUMMARY OF THE INVENTION

An electronic endoscope of the present invention includes: an insertion portion insertable into an inner space of a channel; an optical adapter which is detachably attached to a distal end of the insertion portion, the optical adapter having an illumination portion formed of LED light emission elements; an image-capturing portion for capturing an image of a subject illuminated by the illumination portion; an illumination drive portion for driving the illumination portion; and a drive adjustment portion for adjusting a condition for driving the illumination drive portion.

Other characteristics and benefits of the present invention will be sufficiently apparent from the descriptions below.

### BRIEF DESCRIPTION OF THE DRAWINGS

FIGS. 1 to 4 relate to a first embodiment of the present invention, wherein:

FIG. 1 is an appearance diagram showing an appearance of an endoscope device;

FIG. 2 is a block diagram showing a configuration of the endoscope device of FIG. 1;

FIG. 3 is a view showing a configuration of an LED drive portion of FIG. 2; and

FIG. 4 is a block diagram showing a configuration of a modification example of the endoscope device of FIG. 2.

FIG. 5 is a block diagram showing a configuration of an endoscope device according to a second embodiment of the present invention.

FIGS. 6 and 7 relate to a third embodiment of the present invention, wherein:

FIG. 6 is a block diagram showing a configuration of an endoscope device; and

FIG. 7 is a view showing a configuration of an LED drive portion of FIG. 6.

FIGS. 8 and 9 relate to a fourth embodiment of the present invention, wherein:

FIG. 8 is a block diagram showing a configuration of an endoscope device; and

FIG. 9 is a view showing a configuration of an LED drive portion of FIG. 8.

FIGS. 10 and 11 relate to a fifth embodiment of the present invention, wherein:

FIG. 10 is an appearance diagram showing an appearance of an endoscope device; and

FIG. 11 is a view showing a placement configuration of a fine-adjustment knob of FIG. 10.

FIG. 12 is a block diagram showing a configuration of a conventional endoscope device.

FIG. 13 is a view showing a first modification example of a white LED of FIG. 12.

FIG. 14 is a view showing a second modification example of the white LED of FIG. 12.

FIG. 15 is a view showing a third modification example of the white LED of FIG. 12.

FIG. 16 is a view showing a fourth modification example of the white LED of FIG. 12.

FIG. 17 is a view showing in a general format a modification example of the white LED of FIG. 12.

FIG. 18 is a view describing a transition of emission characteristics of an LED.

### DETAILED DESCRIPTION OF PREFERRED EMBODIMENTS

### First Embodiment

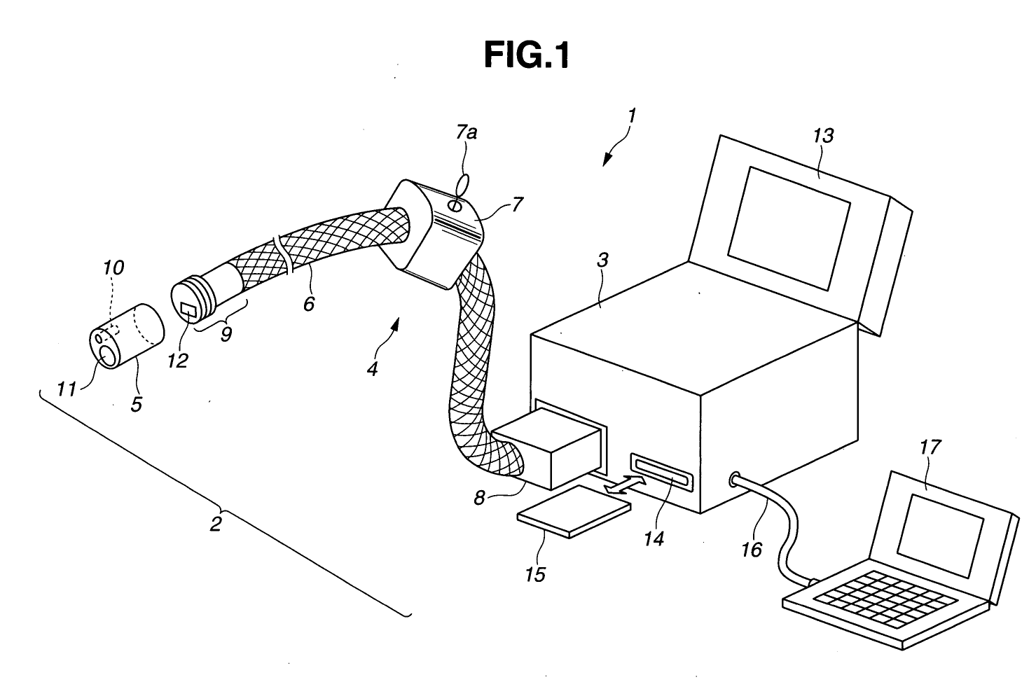

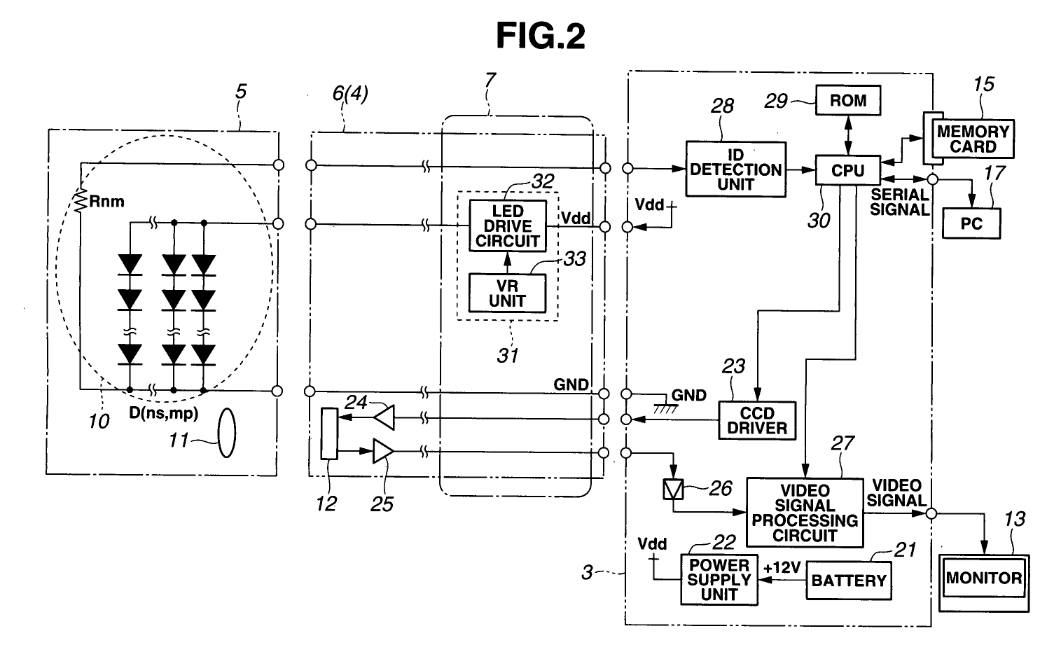

As shown in FIGS. 1 and 2, an endoscope device 1 of the present embodiment includes: an endoscope portion 4 having an elongate and flexible insertion portion 6 which has a bendable bending portion at a distal end side, and a CCD 12 serving as an image-capturing portion in a distal end portion 9 of the insertion portion 6; an optical adapter 5 with a white LED portion 10 built therein serving as an illumination portion, which is detachably and electrically connected to the distal end portion 9 of the endoscope portion 4; and a main body 3 that drives the white LED portion 10 and also signal processes an output signal from the CCD 12 to generate a video signal so as to display an endoscope image on a monitor 13. The endoscope portion 4 and the optical adapter 5 configure an endoscope unit 2. Note that the CCD 12 may be provided in the optical adapter 5.

Also, the insertion portion 6 is provided with an operation portion 7, and by operating a bending operation knob 7a of the operation portion 7, the bending portion of the insertion portion 6 can be bent.

The optical adapter 5 has, in addition to the white LED portion 10, an object optical system 11 for forming, on an image-capturing surface of the CCD 12, an image of a subject irradiated with illumination light from the white LED portion 10.

At a proximal end of the endoscope portion 4, a scope connector 8 is provided which is detachably and electrically connected to the main body 3.

To the main body 3, a memory card 15 can be detachably connected via a card slot 14, and the main body 3 and a personal computer (PC) 17 can be connected via a cable 16.

As shown in FIG. 2, the white LED portion 10 of the optical adapter 5 includes a plurality of LED elements in an n-serial and m-parallel matrix configuration D (nS, mP), and an identification resistance Rnm for identifying the type of the white LED portion 10.

In the operation portion 7 of the endoscope portion 4, an LED drive portion 31 for driving the white LED portion 10 is provided. The LED drive portion 31 is configured by an LED drive circuit 32 serving as an illumination drive portion, and a volume unit (hereinafter abbreviated as “VR unit”) 33 as a drive adjustment portion. The LED drive circuit 32 and the VR unit will be described in detail later.

The CCD 12 of the endoscope portion 4 is driven by a CCD drive signal from a CCD driver 23 of the main body 3 via a buffer 24. An output signal of the CCD 12 is outputted to a preamplifier 26 in the main body 3 via a buffer 25.

The main body 3 includes a video signal processing circuit 27 for signal processing an output signal of the CCD 12 which is amplified by the preamplifier 26 so as to output a video signal to the monitor 13.

In the main body 3, the identification resistance Rnm of the white LED portion 10 is detected by an ID detection unit 28, and with the detection signal, a CPU 30 identifies the type of the white LED portion 10.

Then, the CPU 30 controls various circuits in the device, such as the CCD driver 23 and the video signal processing circuit 27, according to a program pre-stored in a ROM 29.

The main body 3 is configured as a carriable device, in that the main body 3 is supplied with an electric power by a battery 21, and a power supply unit 22 generates an electric power of a circuit voltage Vdd in the device.

Note that in the main body 3, the CPU 30 can transmit data such as an endoscope image to the memory card 15 and the PC 17, thereby saving these data thereinto.

The circuit voltage Vdd of the main body 3 is also outputted to the LED drive circuit 32 of the endoscope portion 4.

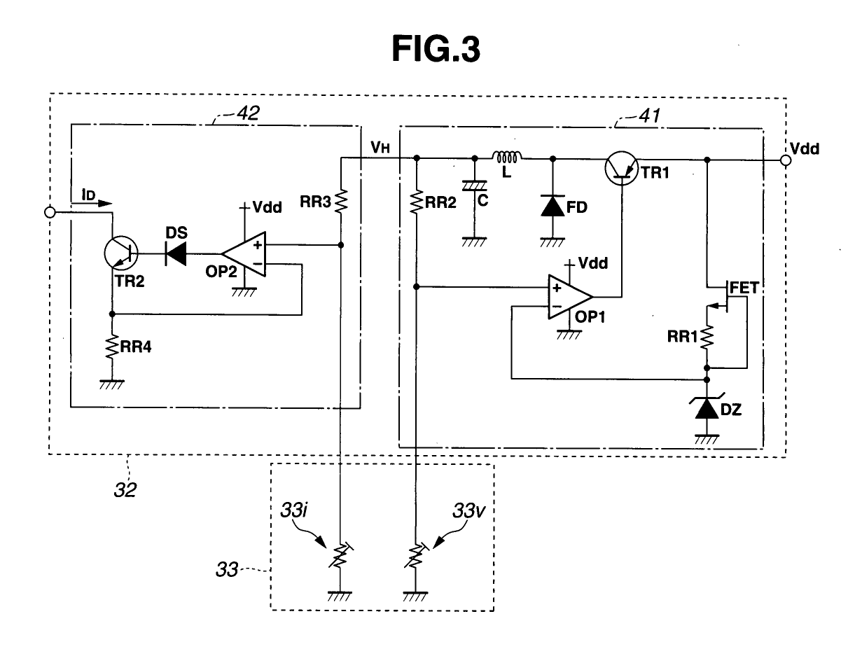

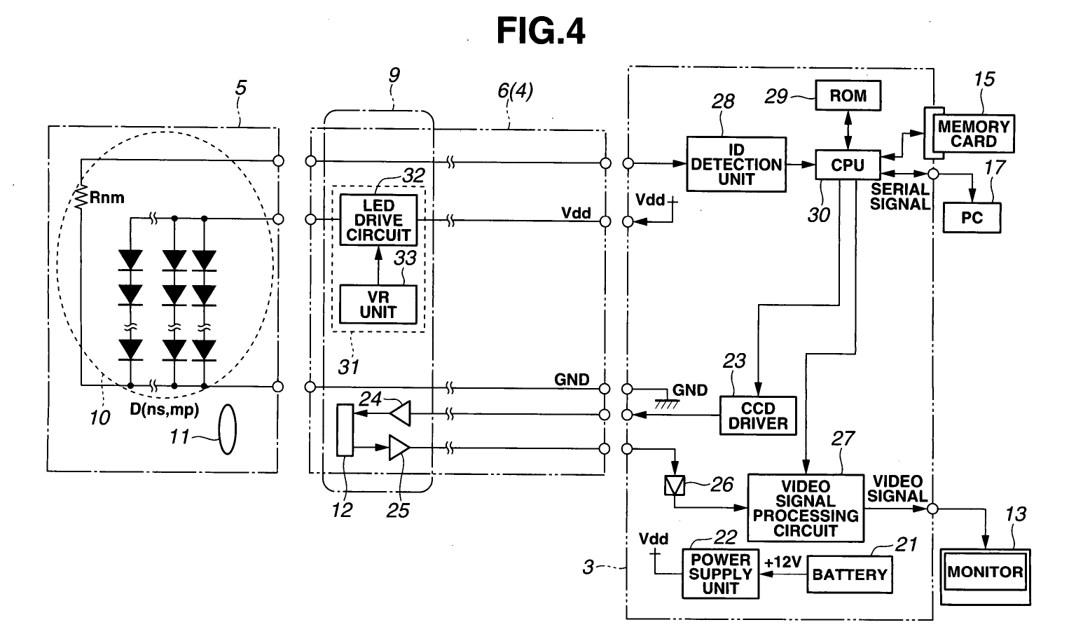

As shown in FIG. 3, the LED drive circuit 32 is configured by a switching circuit 41 of, e.g., voltage step-down type, for switching the circuit voltage Vdd to generate an LED drive voltage VH, and a constant current circuit 42 which uses the LED drive voltage VH as an input voltage to generate an LED drive current ID. The VR unit 33 is configured by a voltage adjusting variable resistance 33v for adjusting the LED drive voltage VH, and a current adjusting variable resistance 33i for adjusting the LED drive current ID. The LED drive voltage VH is adjusted such that VH ≧n ×VF (where VF is light emission voltage of the white LED elements). The switching circuit 41 may be configured as a voltage step-up type circuit.

TR1 is a switching transistor, and FET, RR1, and DZ provide a constant current source to supply a reference voltage to the minus (−) input of OP1. FD is a fly-wheel diode for supplying energy to L and C when the TR1 is switched off. The VH smoothed by the L and C is voltage divided by RR2 and the voltage adjusting variable resistance 33v, and is inputted to OP1 (+).

The OP1 compares the reference voltage of the (−) input and the divided voltage of VH of the (+) input, and from an output of the OP1 supplies an error voltage to the base of the TR1. Fluctuation in the error voltage is feedback controlled to maintain the VH to a constant voltage.

OP2, DS, and TR2 operate as a voltage-current conversion circuit, operating to convert a constant potential inputted to the OP2 (+) to a current value to flow into RR4.

Thus, in this embodiment, in the operation portion 7 of the endoscope portion 4 is provided the LED drive portion 31 including the LED drive circuit 32 and the VR unit 33 for driving the white LED portion 10 of the optical adapter 5. This allows the VR unit 33 of the LED drive portion 31 to adjust the LED drive voltage VH and the LED drive current ID in a corresponding manner to the white LED portion 10.

Therefore, even if the endoscope unit 2 including the endoscope portion 4 and the optical adapter 5 is exchanged according to inspection situations, the white LED portion 10 can emit light under the optimum driving condition, without adjusting the main body 3.

In addition, because the LED drive portion 31 is not provided to the main body 3, not only the main body 3 can be simplified in configuration, but also an inspection becomes possible simply by connecting the endoscope unit 2 having the white LED portion 10 of different configurations, thus allowing for a versatile use of the main body 3 for the endoscope unit 2.

Note that, although the LED drive portion 31 is provided in the operation portion 7 of the endoscope portion 4 in the present embodiment, no limitation is placed thereon, but the LED drive portion 31 may be provided in the distal end portion 9 of the endoscope portion 4 to obtain the same operations and effects. Moreover, the LED drive portion 31 may be provided in the scope connector 8, although this configuration is not shown.

### Second Embodiment

A second embodiment is almost the same as the first embodiment. Therefore, only different points will be described, and the same components are attached with the same symbols and descriptions thereof will be omitted.

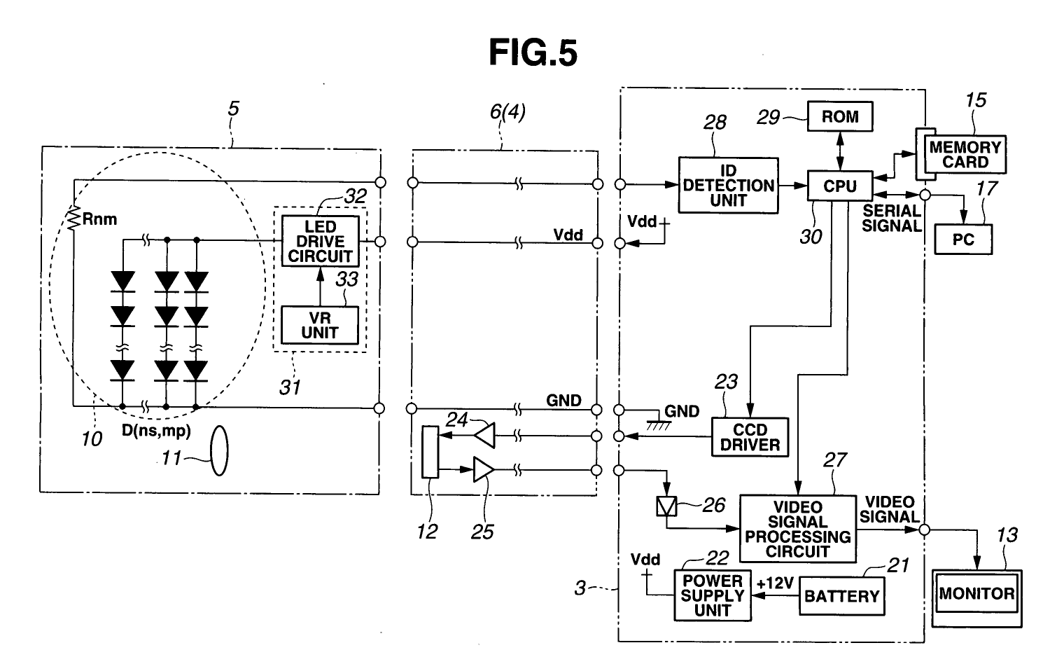

In this embodiment, the LED drive portion 31 is provided in the optical adapter 5, as shown in FIG. 5. Other components and operations are the same as in the first embodiment.

Thus, in this embodiment, in addition to the effects of the first embodiment, the versatility of the endoscope portion 4 and the main body 3 can be increased because the optical adapter 5 alone has a complete configuration of an illumination optics system, in contrast to the first embodiment which enhances the versatility of the main body 3 with respect to the endoscope unit 2.

### Third Embodiment

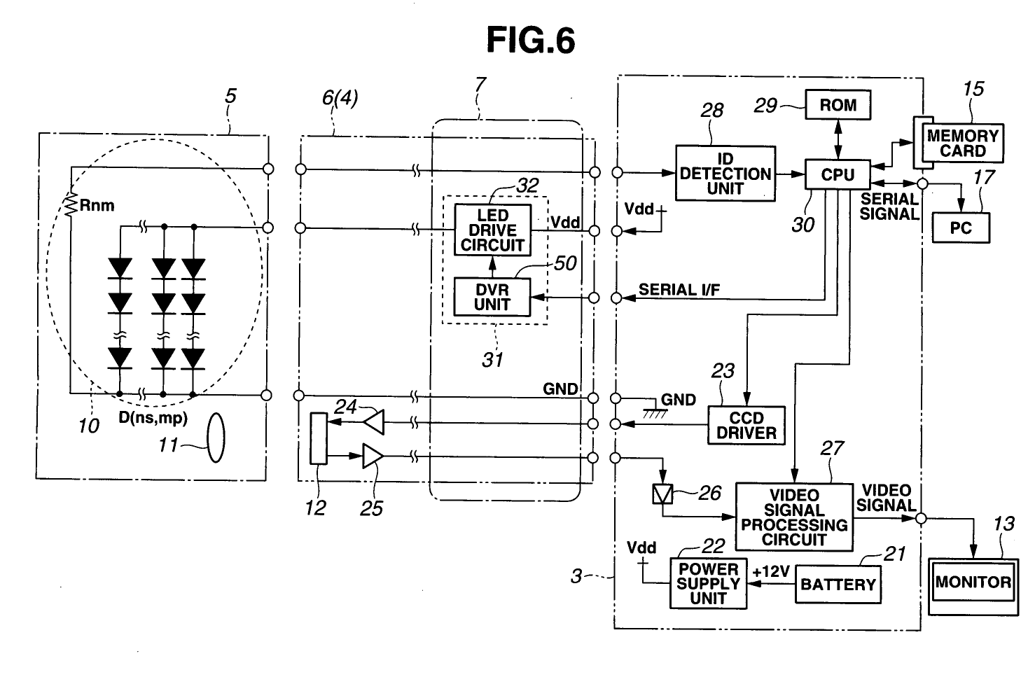

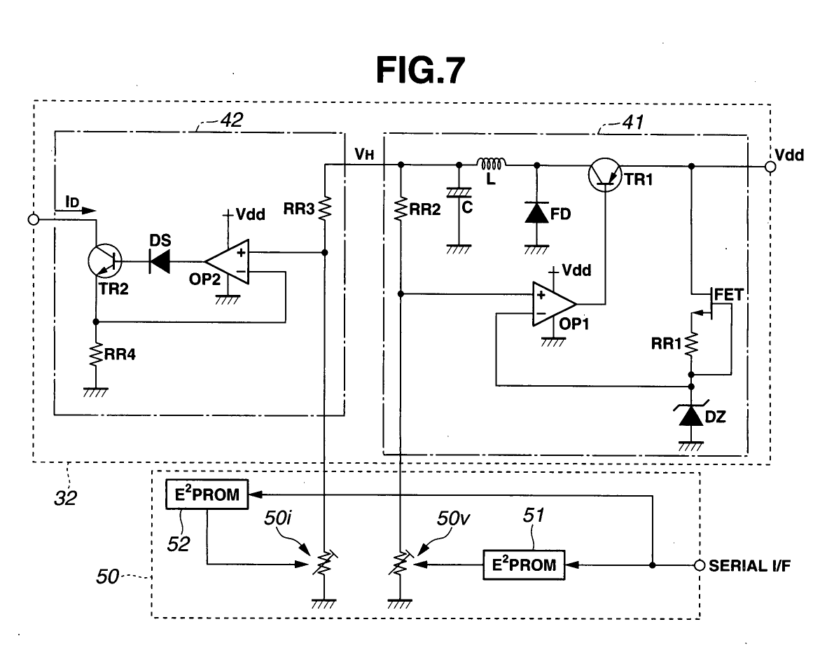

FIGS. 6 and 7 relate to a third embodiment of the present invention. FIG. 6 is a block diagram showing a configuration of the endoscope device, and FIG. 7 is a view showing a configuration of the LED drive portion of FIG. 6.

The third embodiment is almost the same as the first embodiment. Therefore, only different points will be described, and the same components are attached with the same symbols and descriptions thereof will be omitted.

As shown in FIG. 6, the LED drive portion 31 is configured by the LED drive circuit 32 and a digital potention unit (hereinafter abbreviated as “DVR unit”) 50.

As shown in FIG. 7, the DVR unit 50 is configured by a digital potention 50v for adjusting the LED drive voltage VH and a digital potention 50i for adjusting the LED drive current ID capable of digitally adjusting resistance value, and two rewritable E²PROMs 51, 52.

Then, numerical values of the E²PROMs 51, 52 are rewritten by a serial signal from the CPU 30, and resistance values of the digital potentions 50v, 50i are adjusted based on the written data of the E²PROMs 51, 52, so as to adjust the LED drive voltage VH and the LED drive current ID in the LED drive circuit 32. The E²PROMs 51, 52 may be provided as one memory so that the one memory possesses data for the digital potention 50v and data for the digital potention 50i.

The CPU 30 identifies the type of the white LED portion 10 by means of the identification resistance Rnm detected by the ID detection unit 28, and outputs to the DVR unit 50 as a serial signal, an adjustment signal for adjusting the LED drive voltage VH and the LED drive current ID of the white LED portion 10 to the optimum value.

Thus, in this embodiment, in addition to the effects of the first embodiment, the adjustment signal is outputted as a serial signal to the DVR unit 50 of the LED drive portion 31. By means of this serial signal, the LED drive voltage VH and the LED drive current ID of the white LED portion 10 can be automatically adjusted to the optimum value.

### Fourth Embodiment

A fourth embodiment is almost the same as the third embodiment. Therefore, only different points will be described, and the same components are attached with the same symbols and descriptions thereof will be omitted.

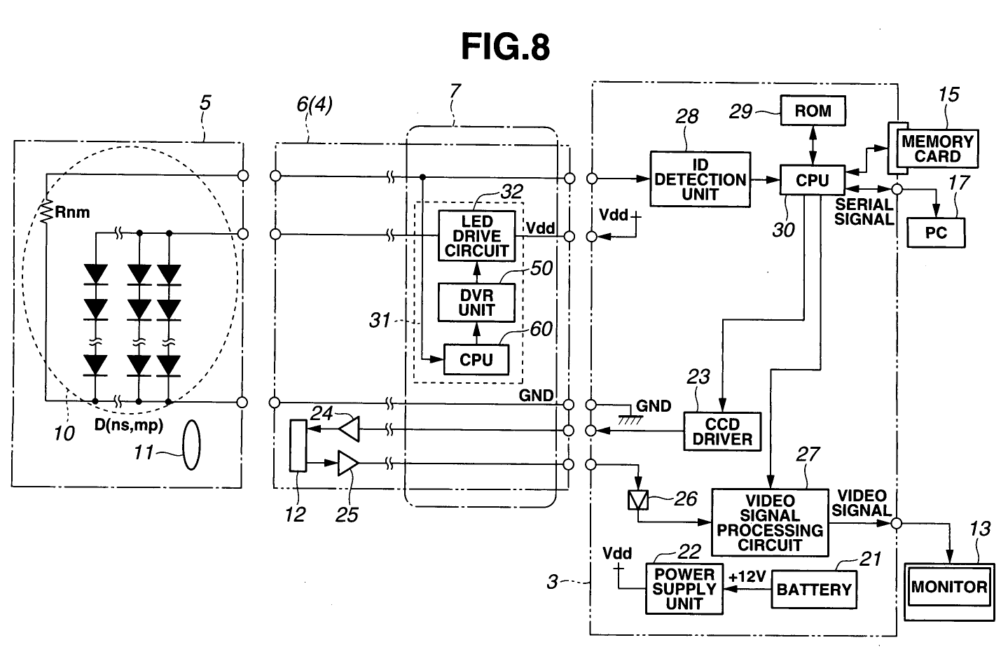

As shown in FIG. 8, the LED drive portion 31 is configured by the LED drive circuit 32, the DVR unit 50, and a CPU 60.

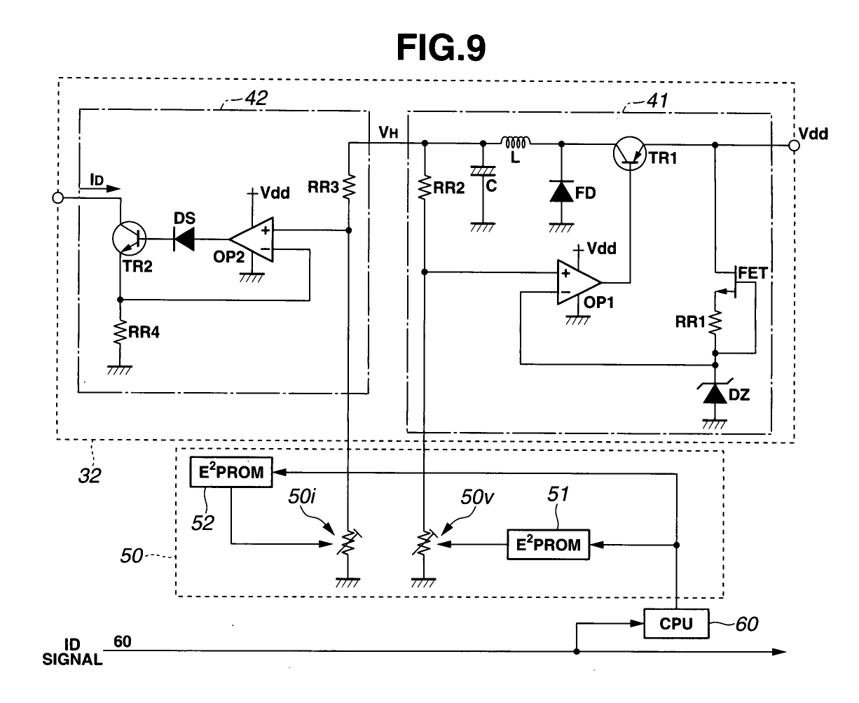

As shown in FIG. 9, similarly to the third embodiment, the DVR unit 50 is configured by the digital potention 50v for adjusting the LED drive voltage VH and the digital potention 50i for adjusting the LED drive current ID capable of digitally adjusting resistance value, and the two rewritable E²PROMs 51, 52. The E²PROMs 51, 52 may be provided as one memory so that the one memory possesses data for the digital potention 50v and data for the digital potention 50i.

Then, numerical values of the E²PROMs 51, 52 are rewritten by a serial signal from the CPU 30, and resistance values of the digital potentions 50v, 50i are adjusted based on the written data of the E²PROMs 51, 52, so as to adjust the LED drive voltage VH and the LED drive voltage ID in the LED drive circuit 32.

The CPU 30 identifies the type of the white LED portion 10 by means of the identification resistance Rnm, and outputs to the E²PROMs 51, 52 as a serial signal, an adjustment signal for adjusting the LED drive voltage VH and the LED drive current ID of the white LED portion 10 to the optimum value.

Thus, the present embodiment can also obtain the same effects as in the third embodiment.

### Fifth Embodiment

A fifth embodiment is almost the same as the first embodiment. Therefore, only different points will be described, and the same components are attached with the same symbols and descriptions thereof will be omitted.

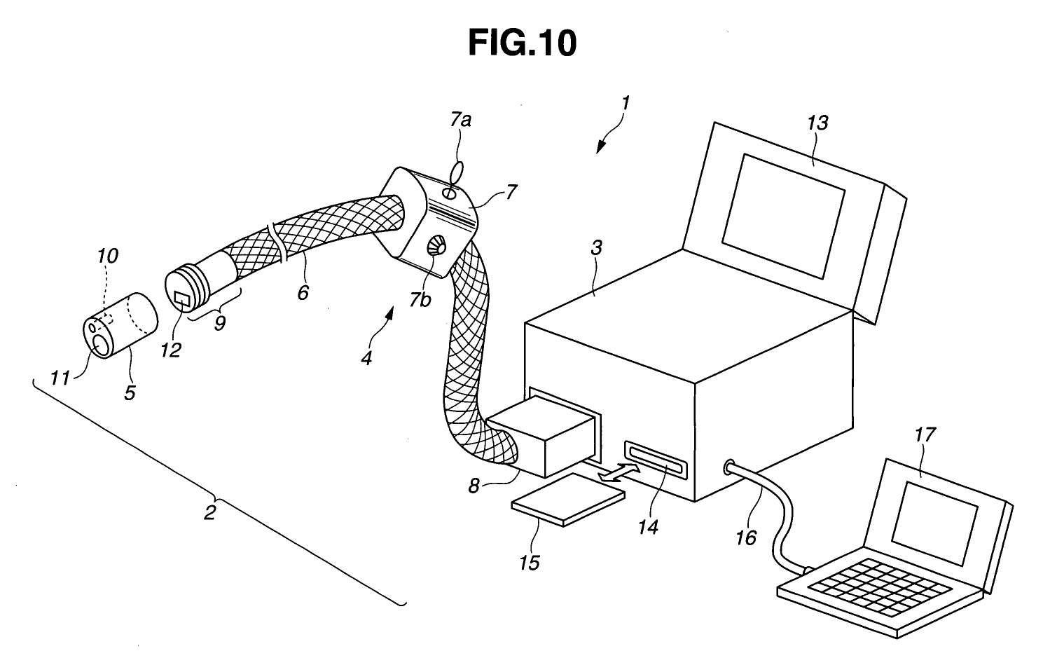

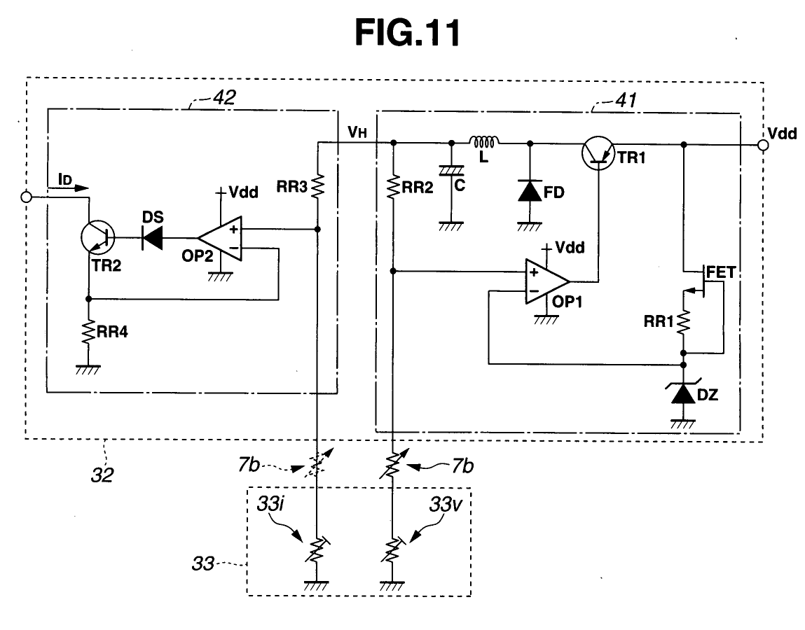

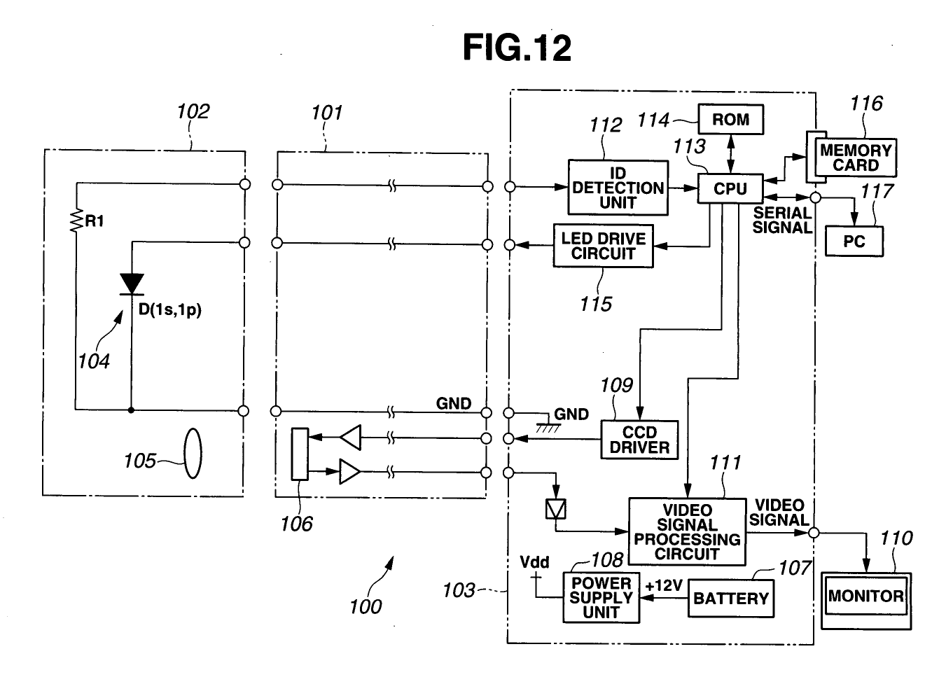

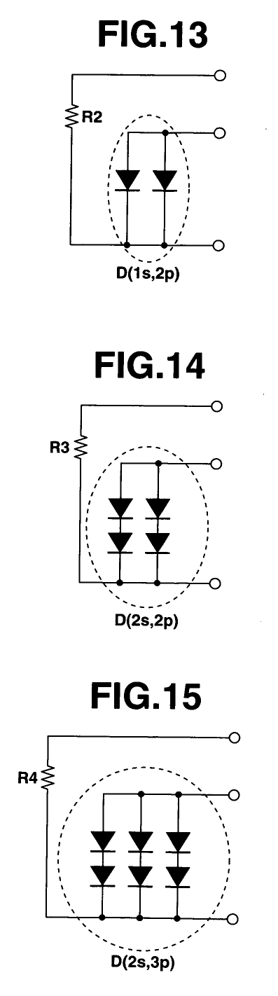

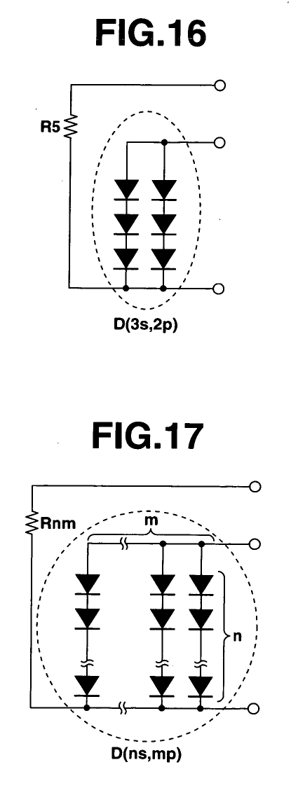

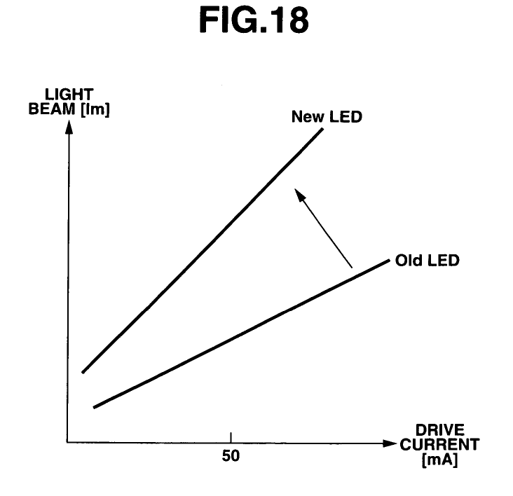

As shown in FIG. 10, the operation portion 7 of the endoscope portion 4 is provided with a brightness fine-adjustment knob 7b for fine-adjusting the emission light amount of the white LED portion 10 and therefore the brightness of the illumination light. As shown in FIG. 11, the brightness fine-adjustment knob 7b is serially connected to the voltage adjusting variable resistance 33v to manually fine-adjust the value of, e.g., the LED drive voltage VH. Other components are the same as in the first embodiment. Note that the brightness fine-adjustment knob 7b may be serially connected to the current adjusting variable resistance 33i as shown with a dotted line in FIG. 11 so that the value of the LED drive voltage ID can be manually fine-adjusted.

Thus, in this embodiment, in addition to the effects of the first embodiment, the brightness of the illumination light can be manually fine-adjusted.

Note that the constant voltage circuit 41 and the constant current circuit 42 of the invention of the present application are realized regardless of the manner in which the description is made.

For example, the constant voltage circuit, which is shown as the switching type, may be a series-type constant voltage circuit (such as three-terminal regulator), or of a circuit type of a simple constant voltage circuit using a Zener diode. Also, the constant current circuit may be of a simple type using a transistor without using an operational amplifier. That is, the circuit may be constituted by any electronic element capable of setting a constant voltage or current value.

In the present invention, it is apparent that embodiments differing in a wide range may be configured based on this invention without departing from the spirit and scope of the invention. The present invention is not restricted by any specific embodiment thereof except of being limited by the appended set of claims.

In the claims:

1. An electronic endoscope, comprising:
an insertion portion insertable into an inner space;
an optical adapter detachably attached to a distal end of the insertion portion, the optical adapter having an illumination portion formed of LED light emission elements;
an image-capturing portion for capturing an image of a subject illuminated by the illumination portion;
an illumination drive portion for driving the illumination portion; and
a drive adjustment portion for adjusting a condition for driving the illumination drive portion.

2. The electronic endoscope according to claim 1, wherein the illumination portion is formed of a plurality of the LED light emission elements.

3. The electronic endoscope according to claim 1, wherein the illumination drive portion is configured by a constant voltage circuit and a constant current circuit.

4. The electronic endoscope according to claim 3, wherein the drive adjustment portion adjusts, as the driving condition, an output value of at least one of the constant voltage circuit and the constant current circuit.

5. The electronic endoscope according to claim 1, wherein the illumination drive portion is placed in the insertion portion.

6. The electronic endoscope according to claim 5, wherein the illumination drive portion is placed in an operation portion provided to the insertion portion.

7. The electronic endoscope according to claim 5, wherein the illumination drive portion is placed in a distal end portion of the insertion portion.

8. The electronic endoscope according to claim 5, wherein the illumination drive portion is placed in a connector portion which is connectable to a signal processing device for signal processing an image-capturing signal from the image-capturing portion, the connector portion being provided to a proximal end of the insertion portion.

9. The electronic endoscope according to claim 1, wherein the illumination drive portion is placed in the optical adapter.

10. The electronic endoscope according to claim 1, further comprising:
an identification portion for identifying a configuration type of the LED light emission elements of the illumination portion; and
a control portion for controlling the drive adjustment portion on the basis of an identification result of the identification portion.

11. An optical adapter attachable to and detachable from a distal end of an insertion portion insertable into an inner space, the optical adapter comprising:
an illumination portion formed of LED light emission elements;
an illumination drive portion for driving the illumination portion; and
a drive adjustment portion for adjusting a condition for driving the illumination drive portion.

12. An endoscope device, comprising:
an electronic endoscope, including:
an insertion portion insertable into an inner space
an optical adapter detachably attached to a distal end of the insertion portion, the optical adapter having an illumination portion formed of LED light emission elements;
an image-capturing portion for capturing an image of a subject illuminated by the illumination portion;
an illumination drive portion for driving the illumination portion; and
a drive adjustment portion for adjusting a condition for driving the illumination drive portion; and
a signal processing portion for signal processing an image-capturing signal from the image-capturing portion to generate an endoscope image.
